(19) United States
(12) Patent Application Publication
(10) Pub. No.: US 2026/0311501 A1
(43) Pub. Date: Oct. 8, 2026

(54) IMPROVED METHOD AND APPARATUS FOR IMAGING OF CELLS AND FINDING CELLS, COUNTING CELLS AND DETERMINING CONFLUENCE OF A CELL CULTURE

(71) Applicant: THRIVE BIOSCIENCE, INC., Beverly, MA (US)

(72) Inventors: Kenneth CHAPMAN, Godley, TX (US); Frank EVANS, Portland, OR (US)

(21) Appl. No.: 19/482,016

(22) PCT Filed: May 6, 2024
(86) PCT No.: PCT/US2024/028021

§ 371 Date: Nov. 6, 2025

Related U.S. Application Data
(60) Provisional application No. 63/464,740, filed on May 8, 2023.

(51) Int. Cl. G06V 20/69; G06V 10/26; G06V 10/34

(52) U.S. Cl. CPC G06V 20/695; G06V 10/26; G06V 10/34

(57) ABSTRACT

A method and apparatus for counting cells in image regions of a phase image of a cell culture in a cell culture vessel, has an imager for imaging a cell culture in a cell culture vessel and at least on processor for providing a phase image of the cell culture and configured to: find the local maximum of each source image region, wherein the size of each source image region is defined by an approximated diameter by creating an intermediate image using an elliptical structuring element mask having a deleted central pixel and performing a morphological operation of dilation on the source image; for each source image region create a new mask image as a logical ‘LARGER THAN’ of the intermediate image with the source image resulting in zero everywhere except at a single pixel dot located at a pixel location wherein the source image has a local maximum value of a local region and wherein the size of the local region is determined by the size of the elliptical structuring element mask resulting in the mask image having a multitude of single pixel dots; create an index image wherein each single non-zero pixel of the mask image is replaced by a unique index value beginning with one and increasing by one for each additional non-zero pixel value; use the index image to create a translation array of source image values, one for each index in the index image, in which the value of the translation array is set to the value contained in the source image at a pixel location identical to the pixel location of each index value of the index image; create a waterfall image using the index image as a seed image causing each seed dot to grow into local regions filled with the seed value until the growth encounters an adjacent region from an adjacent seed resulting in the expansion of the plurality of dots in the index image into a plurality of uniformly filled regions forming a Voronoi-type diagram in which each region is filled with the index value from a seed dot contained within that region; create a local-maximum image by replacing each index value in the waterfall image with its corresponding value from the translation array resulting in a Voronoi-type diagram in which each region is uniformly filled with the maximum value of the source image for each region; create a local threshold value in each of the plurality of regions by multiplying the local-maximum image by a threshold parameter with a range of [0.0 . . . 1.0] to create a local threshold value in each image region; create a segmentation image wherein values are zero except where values in the source image region are larger than the values in the local-threshold image to isolate each local region into a single area which contains a maximum value of the region and any adjacent pixels which are above the local maximum value; create a contour around the edge of each segmented image area; create lists of outlines, wherein each outline has its own outline list of points and shape parameters, wherein each outline indicates the presence of a cell; and count the number of cells.

### PRIORITY CLAIM

This application claims priority of U.S. Provisional Application Ser. No. 63/464,740, filed May 8, 2023, the entire contents of which are hereby incorporated herein by reference.

### FIELD OF THE INVENTION

The present invention relates to imaging methods and apparatus and in particular to the imaging of cell cultures.

### BACKGROUND

Cell culture incubators are used to grow and maintain cells from cell culture, which is the process by which cells are grown under controlled conditions. Cell culture vessels containing cells are stored within the incubator, which maintains conditions such as temperature and gas mixture that are suitable for cell growth. Cell imagers take images of individual or groups of cells for cell analysis.

While scientists use microscopes to observe cells during culturing and may also attach a camera to the microscope to image cells in a cell culture, such imaging systems have many disadvantages and are limited in the information they provide to the scientist.

### SUMMARY

The object of the present invention is to provide an improved imaging method and apparatus for the finding of cells, the counting of cells and the determination of the confluence of a cell culture using a phase image of the cells in a cell culture and in some embodiments, the quantitative phase image of a cell culture. An imaging method and apparatus for use herein is described in U.S. application Ser. No. 15/563,375 filed on Mar. 31, 2016, the disclosure of which in its entirety is hereby incorporated by reference.

Thrive Bioscience. Inc. the assignee hereof, provides methods and apparatus for illumination and capture of brightfield images. The current implementations of this hardware are called the CellAssist® and CellAssist®50 and can be used to carry out some embodiments of the invention. In addition to the CellAssist, examples of other embodiments of imagers capable for implementing embodiments of the present invention are disclosed in applications PCT/US22/45846 filed Oct. 6, 2022 and PCT/US23/11114 filed Jan. 19, 2023, the disclosures of which in their entirety are hereby incorporated by reference.

These and other objects of the present invention are achieved in some embodiments by a method for counting cells in image regions of a phase image, the source image, of a cell culture in a cell culture vessel, comprising the steps of finding the local maximum of each source image region, wherein the size of each source image region is defined by an approximated diameter by creating an intermediate image using an elliptical structuring element mask having a deleted central pixel and performing a morphological operation of dilation on the source image, for each source image region creating a new mask image as a logical ‘LARGER THAN’ of the intermediate image with the source image resulting in zero everywhere except at a single pixel dot located at a pixel location wherein the source image has a local maximum value of a local region and wherein the size of the local region is determined by the size of the elliptical structuring element mask resulting in the mask image having a multitude of single pixel dots; creating an index image wherein each single non-zero pixel of the mask image is replaced by a unique index value beginning with one and increasing by one for each additional non-zero pixel value, using the index image to create a translation array of source image values, one for each index in the index image, in which the value of the translation array is set to the value contained in the source image at a pixel location identical to the pixel location of each index value of the index image; creating a waterfall image using the index image as a seed image causing each seed dot to grow into local regions filled with the seed value until the growth encounters an adjacent region from an adjacent seed resulting in the expansion of the plurality of dots in the index image into a plurality of uniformly filled regions forming a Voronoi-type diagram in which each region is filled with the index value from a seed dot contained within that region; creating a local-maximum image by replacing each index value in the waterfall image with its corresponding value from the translation array resulting in a Voronoi-type diagram in which each region is uniformly filled with the maximum value of the source image for each region; creating a local threshold value in each of the plurality of regions by multiplying the local-maximum image by a threshold parameter with a range of [0.0 . . . 1.0] to create a local threshold value in each image region; creating a segmentation image wherein values are zero except where values in the source image region are larger than the values in the local-threshold image to isolate each local region into a single area which contains a maximum value of the region and any adjacent pixels which are above the local maximum value; creating a contour around the edge of each segmented image area, creating lists of outlines, wherein each outline has its own outline list of points and shape parameters, wherein each outline indicates the presence of a cell and counting the number of cells.

In some embodiments, the method further comprises finding colonies of cells by counting the segmented outlines, enlarge each segmented region, resegmenting, and count outlines again, repeating the enlarge and count steps until the total number of segmented outlines drops to a desired percentage of the starting count value, finding contours that remain and determine the outline(s) of the region(s) which delineate where large colonies of cells are congregated, wherein the area enclosed by the outline(s) of the congregated colonie(s) are measured and compared to the total area of the subsection region to determine confluence. In some embodiments the desired percentage is 50%.

These and other objects of the invention are also achieved in some embodiments in accordance with an apparatus for counting cells in image regions of a phase image of a cell culture in a cell culture vessel, comprising: an imager for imaging a cell culture in a cell culture vessel; and at least on processor for providing a phase image, the source image, of the cell culture and configured to: find the local maximum of each source image region, wherein the size of each source image region is defined by an approximated diameter by creating an intermediate image using an elliptical structuring element mask having a deleted central pixel and performing a morphological operation of dilation on the source image; for each source image region create a new mask image as a logical ‘LARGER THAN’ of the intermediate image with the source image resulting in zero everywhere except at a single pixel dot located at a pixel location wherein the source image has a local maximum value of a local region and wherein the size of the local region is determined by the size of the elliptical structuring element mask resulting in the mask image having a multitude of single pixel dots; create an index image wherein each single non-zero pixel of the mask image is replaced by a unique index value beginning with one and increasing by one for each additional non-zero pixel value; use the index image to create a translation array of source image values, one for each index in the index image, in which the value of the translation array is set to the value contained in the source image at a pixel location identical to the pixel location of each index value of the index image; create a waterfall image using the index image as a seed image causing each seed dot to grow into local regions filled with the seed value until the growth encounters an adjacent region from an adjacent seed resulting in the expansion of the plurality of dots in the index image into a plurality of uniformly filled regions forming a Voronoi-type diagram in which each region is filled with the index value from a seed dot contained within that region; create a local-maximum image by replacing each index value in the waterfall image with its corresponding value from the translation array resulting in a Voronoi-type diagram in which each region is uniformly filled with the maximum value of the source image for each region; create a local threshold value in each of the plurality of regions by multiplying the local-maximum image by a threshold parameter with a range of [0.0 . . . 1.0] to create a local threshold value in each image region; create a segmentation image wherein values are zero except where values in the source image region are larger than the values in the local-threshold image to isolate each local region into a single area which contains a maximum value of the region and any adjacent pixels which are above the local maximum value; create a contour around the edge of each segmented image area; create lists of outlines, wherein each outline has its own outline list of points and shape parameters, wherein each outline indicates the presence of a cell; and count the number of cells.

In some embodiments, the at least one processor is further configured for finding colonies of cells by counting the segmented outlines, enlarge each segmented region, resegmenting, and count outlines again; repeat the enlarging and counting until the total number of segmented outlines drops to a desired percentage of the starting count value; find contours that remain and determine the outline(s) of the region(s) which delineate where large colonies of cells are congregated, wherein the area enclosed by the outline(s) of the congregated colonie(s) are measured and compared to the total area of the subsection region to determine confluence. In some embodiments the desired percentage is 50%.

These and other objects and advantages of the present invention will be explained in more detail with respect to the attached drawings wherein:

### BRIEF DESCRIPTION OF THE DRAWINGS

FIG. 1 is a block diagram of an apparatus for carrying out some embodiments of the method and apparatus of the invention;

FIG. 2A shows a typical well of a cell culture vessel;

FIG. 2B shows a top view of the tiling of images of the cell culture vessel well;

FIG. 3 shows the Z-stack of images for a single tile of FIG. 1B;

FIG. 4A shows the two lowest Z stack focal planes with one pixel highlighted;

FIG. 4B shows the voxel corresponding to the highlighted pixel in FIG. 3A;

FIG. 5 is a sample intensity image;

FIG. 6 is the QPI image corresponding to FIG. 4;

FIG. 7 is a colorized version of the FIG. 5 image;

FIG. 8 is a section of the colony contour at the mask edge and

FIG. 9 is one embodiment of a structuring element kernel for use in finding cells;

FIG. 10 is one embodiment of a phase image processed with the kernel of FIG. 9; and

FIG. 11 is the image of FIG. 10 after having a watershed algorithm applied thereto.

### DETAILED DESCRIPTION

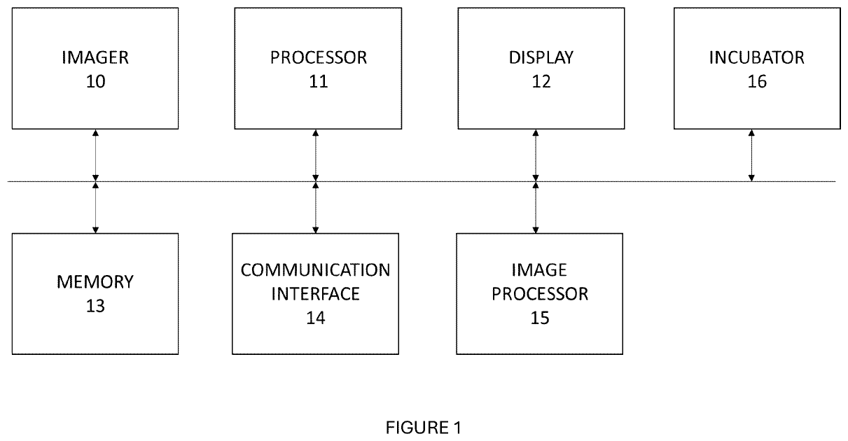

The apparatus in FIG. 1, which shows one embodiment of the present invention, includes an imager 10 and an incubator 16 run by processor 11 which is a microcontroller or microprocessor which has associated RAM and ROM memory 13 for storage of firmware and data. A connection through communication interface 14 allows connection to an external computer. The display 12 acts as a user interface and image processing can take place locally on image processor 15 or it can take place remotely using a more a powerful processor and more image storage to be available via the communication interface 14.

The imager is for example, the CellAssist which generates a stack of brightfield images covering a range of focus planes. This stack of images, referred to as a z-stack, enables processing the information in ways not available for a single image.

One such process allows for the selection of the best or central focus image. It also allows processing of above and below focus image pairs to find the best focal plane.

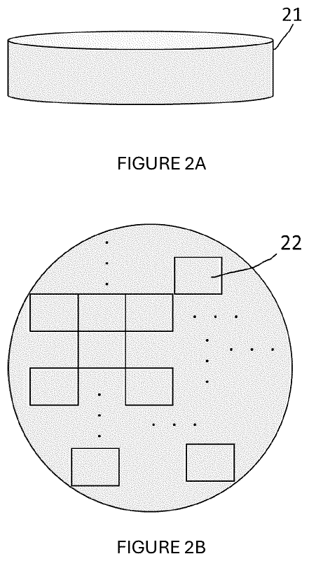

When the well 21 in FIG. 2A of the cell culture vessel is larger than the area imaged by the imager optics, the imager breaks up the image of the cell culture into a plurality of image tiles.

FIG. 2B shows a number of tile images 22. The image processor then stitches the image tiles together to form an image of the culture in the whole well.

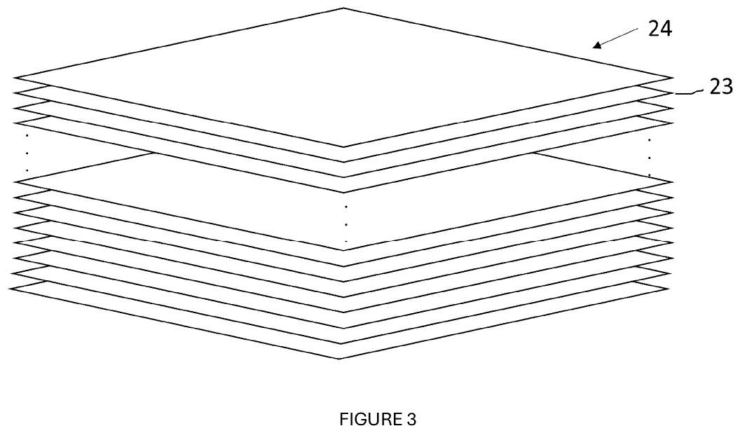

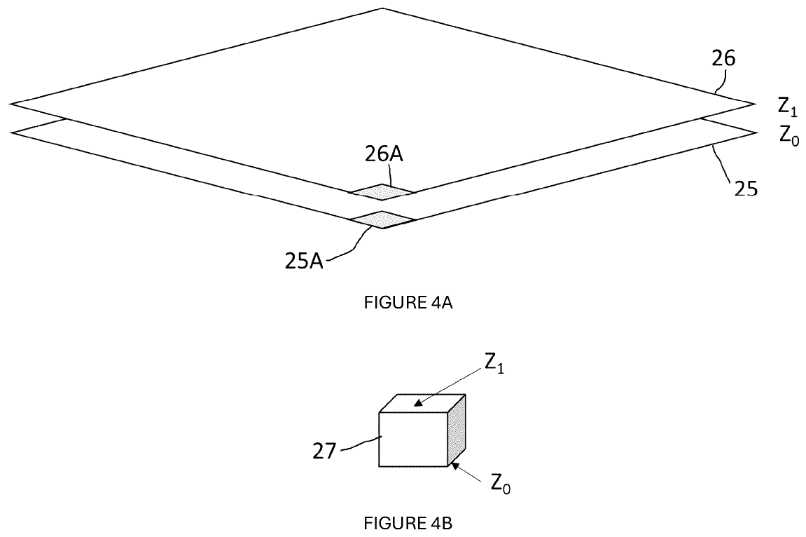

FIG. 3 illustrates a z-stack 24 of tile images 23. The number of z planes can range from 4 to 100 in number per tile, or in some embodiments from 7-23. FIG. 4A shows two z planes z₀ and z₁ which are 25 and 26 respectively with one pixel 25A and 26A in each highlighted for purposes of discussion and which is not shown to scale. In a device like the CellAssist, the number of pixels is 1944×2592 which is approximately 5 megapixels in number. In FIG. 4B, the voxel 27 defined by the pixels in the two adjacent planes z₀ and z₁ is illustrated, also not to scale.

Illumination passes through a subject cells to a camera lens. “Below focus” describes a plane that is closer to the illuminator than the subject plane and “above focus” describes a plane that is farther away from the illuminator than the subject plane. The “best focus image” is the image of the subject plane.

In some embodiments, each whole well image is the result of the stitching together of a number of tiles. The number of tiles needed depend on the size of the well and the magnification of the camera objective. For example, a single well in a 6-well plate is the stitched result of 35 tiles from a 4× camera, 234 tiles from a 10× camera, or 875 tiles from a 20× camera.

The higher magnification objective cameras have smaller optical depth, that is, the z-height range in which an object is in focus. To achieve good focus at higher magnification, a smaller z-offset is preferred. As the magnification increases, the number of z-stack images increases or the working focal range decreases. If the number of z stack images increase, more resources are required to acquire the image, including time, memory and processing power. If the focal range decreases, the likelihood that the cell images will be out of focus is greater, due to instrument calibration accuracy, cell culture plate variation, well coatings, etc.

In some embodiments, the starting z-height value is determined by a database value assigned stored remotely or in local RAM. The z-height is a function of the cell culture plate type and manufacturer and is the same for all instruments and all wells. Any variation in the instruments, well plates, or coatings needs to be accommodated by a large number of z-stacks to ensure that the cells are in the range of focus adjustment. In practice this results in large imaging times and is intolerant to variation, especially for higher magnification objective cameras with smaller depth of field. For example, the 4× objective camera takes 5 z-stack images with a z-offset of 50 μm for a focal range of 5*50=250 μm. The 10× objective camera takes 11 z-stack images with a z-offset of 20 μm for a focal range of 11*20=220 μm. The 20× objective camera takes 11 z-stack images with a z-offset of 10 μm for a focal range of 11*10=110 μm.

Phase is an important component of an optical wavefield providing information on the refractive index, optical thickness, and the topology of the specimen. Phase retrieval is a challenge since the phase of a wavefield is not accessible directly. Phase contrast imaging adds complexity to an illumination system and requires careful selection of apertures to capture the edge changes of phase. In contrast, it is relatively easy to measure the intensity of the wavefield in a brightfield image.

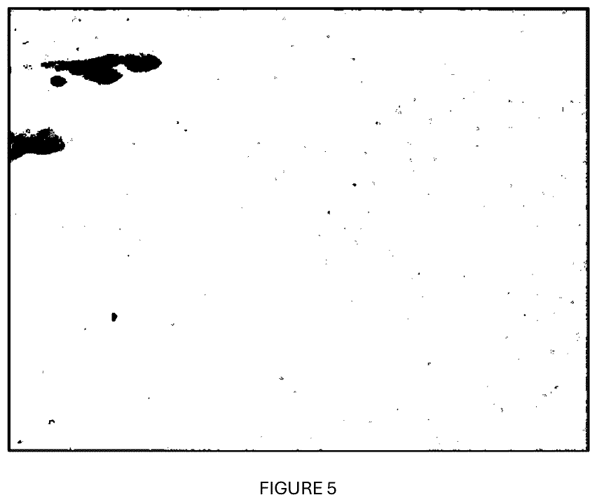

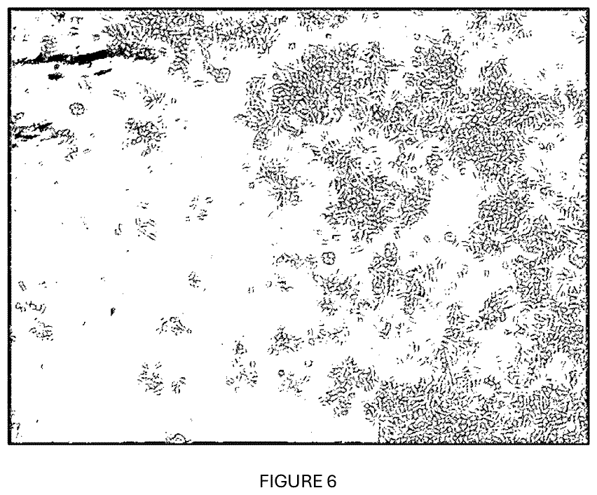

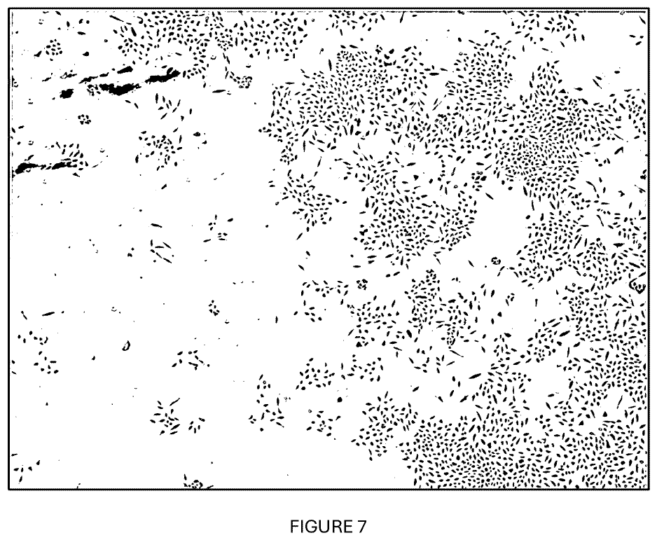

FIG. 5 is a sample intensity brightfield image of a cell culture. The results of the quantitative phase image (QPI) of this intensity brightfield image is shown in FIG. 6. FIG. 7 is a colorized version of the FIG. 6 image which makes the identification of cells even clearer.

The overall method presented here first finds the local maximum of regions of “cell size”. It then creates a local threshold value in each cell sized region. This threshold value is then used to create a mask which separates each cell. The threshold is separate for each cell enabling a reliable contour to be created around the edge of each detected cell phase region. Diagnostic parameters are derived from these contour shapes.

In the mask of detected cells, the process guarantees that none of the cells touch an adjacent cell. To detect colonies of cells, we count the disconnected regions (initially the cells). Then we enlarge each isolated region and count regions again. We continue to do this enlarge/count operation until the total number of detected regions drops to a desired percentage of the starting count value. This decrease indicates that the closely packed, detected cells that did not overlap with adjacent cells, have now expanded to touch the neighboring cells. It is then possible to find the contour(s) that remain and thus determine the outline of the regions where large colonies of cells are congregated. The area enclosed by the outlines of these congregations are measured and compared to the total area of the analysis region to determine the Confluence value (range [0.0 . . . 1.0]). A section of the colony contour at the colony edge and with some ‘holes’ is illustrated with a green contour in FIG. 8. The Cells are outlined in blue and cell centers are the yellow dots.

The process defined here is able to detect cells in phase images and provide derived measurements such as cell count, confluence, and output data that can be further examined for shape and other properties. The height clarity of cells in the phase images enables detection that is less sensitive to artifacts that normally interfere with the reliable segmentation of single cells in unstained cultures.

The ability to do this analysis in unstained cultures enables maintaining cultures which remain alive.

In accordance with the invention, one embodiment of the method for counting cells in image regions of a phase image of a cell culture in a cell culture vessel includes:
a. find the local maximum of cell size of each image region;
b. create a local threshold value in each cell-sized image region;
c. create a mask using the threshold value which separates each cell to segment the cells into cell phase regions;
d. create a contour around the edge of each segmented cell phase region to define a cell outline;
e. create lists of the cell outlines, wherein each cell has its own outline list of points and shape parameters; and
f. count the number of cells.

In some embodiments the lists are used to count the number of cells. In some embodiments, the method further comprises finding colonies of cells by counting the segmented cell phase regions to produce a starting count value, enlarging each segmented cell phase region and counting segmented cell phase regions again; repeating the enlarging and counting segmented cell phase region steps until the total number of segmented cell phase regions drops to a desired percentage of the starting count value, finding contours that remain, determining the outline of regions where large colonies of merged cells are congregated, measuring an area enclosed by the outlines of the congregated colonies and comparing the area to a total area of the image region to determine confluence. In some embodiments, the desired percentage is 50%.

In accordance with the invention, one embodiment of the apparatus for counting cells in image regions of a phase image of a cell culture in a cell culture vessel includes:
an imager for producing images of a cell culture in a cell culture vessel, processing those images to create a phase image; and at least one processor configured to process image regions of the phase image to:
a. find the local maximum of cell size of each image region;
b. create a local threshold value in each cell-sized image region;
c. create a mask using the threshold value which separates each cell to segment the cells into cell phase regions;
d. create a contour around the edge of each segmented cell phase region to define a cell outline;
e. create lists of the cell outlines, wherein each cell has its own outline list of points and shape parameters; and
f. count the number of cells.

In some embodiments the lists are used to count the number of cells. In some embodiments of the apparatus the at least one processor is further configured to find colonies of cells by counting the segmented cell phase regions to produce a starting count value, enlarging each segmented cell phase region and counting segmented cell phase regions again; repeating the enlarging and counting the segmented cell phase regions steps until the total number of segmented cell phase regions drops to a desired percentage of the starting count value, finding contours that remain, determining the outline of regions where large colonies of merged cells are congregated, measuring an area enclosed by the outlines of the congregated colonies and comparing the area to a total area of the image region to determine confluence. In some embodiments, the desired percentage is 50%.

As a result of obtaining the phase image of FIG. 6 of the cell culture, embodiments of the invention herein are improvements in the method and apparatus for counting live cells in the image and estimating confluence.

This document describes a method of processing micrographs which then leads to the ability to count live cell objects in the image.

In some embodiments the source images for this process are phase images which are created from the focus stack and represent the local phase differences of the cells in the culture. In this description, the image of FIG. 6 will be used. This image is the local phase difference scaled and offset to unsigned 8-bit values. It presents the zero phase difference at value 16 and most of the cell phase changes are values above this ‘floor’.

The overall method presented here first finds the local maximum of regions of “cell size” (a CLI parameter). It then creates a local threshold value in each cell sized region. This threshold value is then used to create a mask which separates each cell. The threshold is separate for each cell but scalable by a CLI parameter enabling a reliable contour to be created around the edge of each detected cell phase region. Descriptive metrics are derived from these contour shapes. An annotated image can be output that shows (optionally) the cell centers, contours, and the colonie(s) that have been detected as well as text of the cell counts and the calculated confluence. The image name, cell count and the confluence are piped to a file in JSON format. In addition, output data for the set of cells as a JSON file can be generated. Details of these steps are elaborated below.

The cells manifest as large concentrated phase changes due to the localized fluids of higher index of refraction. The result is that each cell contains one or more smooth peaks of phase. In the phase image, these peaks can be detected with a simple morphology operation.

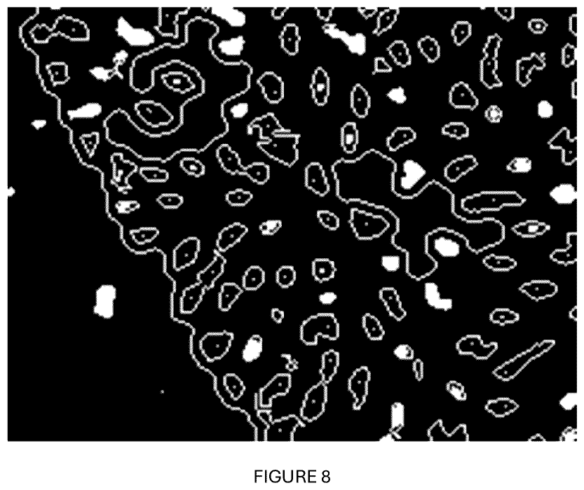

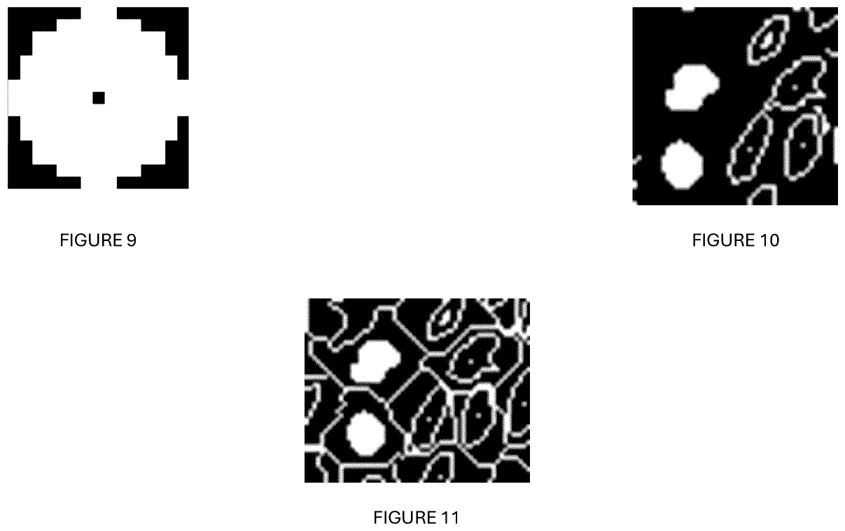

First, define a structuring kernel with a diameter approximating the diameter of the centers of the cells to be detected. For example, the kernel in FIG. 9 is 15×15 pixels and is generated as elliptical and nearly round.

The purpose of the central dot will be explained below. For the moment, conceive the use of this kernel to dilate or enlarge the phase image to create a new image. The result of the dilate operation is a new image composed of the phase values which are the maximum phase values locally covered by this kernel. The entire output image will have values which are larger than or equal to the phase image (ignoring the influence of the central “hole”).

Now consider the effect of the central “hole” in this “donut” mask. When the kernel is positioned with this hole exactly over the local maximum of the local region, the output of this dilate operation will be the second largest value of the region. This is because the hole says, “Do NOT consider the value at that location”, when determining the maximum. The entire image (considering the hole) will now have values which are larger than or equal to the phase image except at the local actual peak value location where the output value of the dilate operation will be LESS than the peak value.

Now create an output peak mask at which:

$$peakMask = new\_image < phaseImg$$

This mask will be all false everywhere except at single pixel dots at the local peak phase value locations.

We can now get a new image, peaks, which contains the phase image values at these peak locations and zero elsewhere by using the and operation (&):
peaks=phaseImg & peakMask

Note: The radius of this kernel should be less than the expected center to center distance between cell phase maximums or the operator will consider the adjacent ‘peaks’ to be a ‘saddle mountain’ and it will detect only the larger peak. An example is shown in FIG. 10.

This operation is scale sensitive. The CLI parameter-scale <scaleAdjust> is provided to manipulate this parameter. The default is 1.0. The kernel, as scaled, is forced to be an odd integer and it is small and the control of this scaling is coarse (i.e., 17×17, 15×15, 13×13, etc.).

This detector will find peak values in nearly empty regions. For this reason, the image of peak center values is thresholded at a small phase value to eliminate these spurious noise detections in empty regions. There is a minimum phase intensity that this detector can accommodate.

The phase image is very conducive to the peak find method defined above. With this high reliability definition for potential cell locations, we can synthesize a local threshold for each cell based on the peak value of its own local phase region. This allows us to create shape outlines for very bright cells and very dim cells with the same ease and reliability. FIG. 10 illustrates this capability.

To define a local threshold, we first use a process called connectedComponents( ) on the peakMask which contains only the points of maximum phase (i.e., yellow dots in FIG. 10). An output image called ‘labels’ is created which replaces the dots of the mask with a unique integer called a label with values in the series [1, 2, 3, 4, . . . .]. This set of labeled values will then be used as the seed image for the watershed algorithm (see below). First however, we pass through the labeled image once to create a lookup table of phase values for each of the labels. The value at index [0] is set to the largest possible phase value. The other table locations, indexed by [labels+1], are set to the detected max phase at the labeled location. Each labeled seed now has a known max phase value preserved in this table.

The Watershed process accepts the labels image and uses the labeled dots as seed values. The watershed then grows these seed dots out over the image until they encounter a non-zero region. Normally, an image defining a surface or ‘terrain’ is provided to the watershed algorithm and the seed growth is halted when this surface is encountered, hence the name “watershed”. If we do not provide a ‘terrain’ image but instead provide a flat image as the ‘terrain’ for the watershed, each seed grows until it encounters its nearest neighbors. The result is a Voronoi diagram of regions defined for each seed. The boundaries are given a special label of (−1). These boundaries are shown as light blue in FIG. 11. The cell outlines are blue, and the phase maximum locations are yellow dots.

If a mask is provided on the CLI command line, this mask is used as the terrain. This limits the local regions to be within the region considered by the mask.

The Voronoi diagram is populated by label values or −1. We add one to the image giving the boundary regions the value 0 and the regions contain [label+1]. The array of max values created above has a special value at index 0 which is larger than any phase value. We now remap the Voronoi image using the label values as an index into this table. We replace the image value (labels) with the stored maxValue of phase. The result is an image where the local region contains the maxValue of the detected phase peak with a very large value at the region boundaries.

The cell outlines can now be segmented from the background. The image above is multiplied by a threshold value (0.0 . . . 1.0] to create a local intensity threshold:

$$Thresh = ( localPhaseMax * threshold )$$

Typical values for threshold are around 0.5 to give a detection of the cell that shows the cell shape well. There is no danger that these cell detections will intersect with neighboring cells because each region of the threshold image is bounded by a very large value at the region edges. Each cell is thresholded at a contour which is a percentage of its own peak value. This makes the shape parameters comparable between cells of widely varying peak intensity.

Once the cells have been segmented, the FindContours( ) procedure can be called to create lists of the cell outlines (shown in Blue in FIG. 10). Each cell has its own outline list of points and shape parameters can be created from this data. This data is used to count the number of cells detected. The entire set of data for these detected cells, including the contour lists and derived shape parameters, can be output as a JSON file.

In the mask of detected cells, the process above guarantees that none of the cells touch an adjacent cell. Cultures organize into local groups of cells, colonies, separated by empty spaces.

To detect colonies of cells, we count the total number of isolated cells in the image. We then dilate the image, enlarging each of the isolated cells, and count again. We continue to do this dilate/count operation until the total number of detected regions drops to a desired percentage of the starting count value (or 10 iterations whichever comes first). This decrease indicates that the closely packed, detected cells that did not initially overlap with adjacent cells, have now expanded to touch the neighboring cells. It is then possible to again invoke the FindContours( ) process and determine the outline of the regions where large colonies of cells are congregated. The area enclosed by the outlines of these congregations are measured and compared to the total area of the masked region to determine the Confluence value (range [0.0 . . . 1.0]. A section of the colony contour at the colony edge and with some ‘holes’, is illustrated with a green contour in FIG. 8.

The process defined here is able to detect cells in phase images and provide derived measurements such as cell count, and confluence. Additional output data is provided that can be further examined for shape and other properties. The high clarity of cells in the phase images enables detection that is less sensitive to artifacts that normally interfere with the reliable segmentation of single cells in unstained cultures.

The ability to do this analysis in unstained cultures enables maintaining and studying cultures which remain alive.

Accordingly, in one embodiment a method for counting cells in image regions of a phase image of a cell culture in a cell culture vessel, comprising the steps of:
a. find the local maximum of each source image region, wherein the size of each source image region is defined by an approximated diameter by creating an intermediate image using an elliptical structuring element mask having a deleted central pixel and performing a morphological operation of dilation on the source image;
b. for each source image region create a new mask image as a logical ‘LARGER THAN’ of the intermediate image with the source image resulting in zero everywhere except at a single pixel dot located at a pixel location wherein the source image has a local maximum value of a local region and wherein the size of the local region is determined by the size of the elliptical structuring element mask resulting in the mask image having a multitude of single pixel dots;
c. create an index image wherein each single non-zero pixel of the mask image is replaced by a unique index value beginning with one and increasing by one for each additional non-zero pixel value;
d. using the index image to create a translation array of source image values, one for each index in the index image, in which the value of the translation array is set to the value contained in the source image at a pixel location identical to the pixel location of each index value of the index image;
e. create a waterfall image using the index image as a seed image causing each seed dot to grow into local regions filled with the seed value until the growth encounters an adjacent region from an adjacent seed resulting in the expansion of the plurality of dots in the index image into a plurality of uniformly filled regions forming a Voronoi-type diagram in which each region is filled with the index value from a seed dot contained within that region;
f. create a local-maximum image by replacing each index value in the waterfall image with its corresponding value from the translation array resulting in a Voronoi-type diagram in which each region is uniformly filled with the maximum value of the source image for each region;
g. create a local threshold value in each of the plurality of regions by multiplying the local-maximum image by a threshold parameter with a range of [0.0 . . . 1.0] to create a local threshold value in each image region;
h. create a segmentation image wherein values are zero except where values in the source image region are larger than the values in the local-threshold image to isolate each local region into a single area which contains a maximum value of the region and any adjacent pixels which are above the local maximum value;
i. create a contour around the edge of each segmented image area;
j. create lists of outlines, wherein each outline has its own outline list of points and shape parameters, wherein each outline indicates the presence of a cell; and
k. count the number of cells.

In some embodiments the method further comprises finding colonies of cells by counting the segmented outlines, enlarge each segmented outline and count outlines again; repeat the enlarge and count steps until the total number of segmented outlines drops to a desired percentage of the starting count value; find contours that remain and determine the outline(s) of the region(s) which delineate where large colonies of cells are congregated, wherein the area enclosed by the outline(s) of the congregated colonie(s) are measured and compared to the total area of the subsection region to determine confluence.

In one embodiment an apparatus for counting cells in image regions of a phase image of a cell culture in a cell culture vessel, comprises: an imager for imaging a cell culture in a cell culture vessel; and at least on processor for providing a phase image of the cell culture and configured to:
a. find the local maximum of each source image region, wherein the size of each source image region is defined by an approximated diameter by creating an intermediate image using an elliptical structuring element mask having a deleted central pixel and performing a morphological operation of dilation on the source image;
b. for each source image region create a new mask image as a logical ‘LARGER THAN’ of the intermediate image with the source image resulting in zero everywhere except at a single pixel dot located at a pixel location wherein the source image has a local maximum value of a local region and wherein the size of the local region is determined by the size of the elliptical structuring element mask resulting in the mask image having a multitude of single pixel dots;
c. create an index image wherein each single non-zero pixel of the mask image is replaced by a unique index value beginning with one and increasing by one for each additional non-zero pixel value;
d. using the index image to create a translation array of source image values, one for each index in the index image, in which the value of the translation array is set to the value contained in the source image at a pixel location identical to the pixel location of each index value of the index image;
e. create a waterfall image using the index image as a seed image causing each seed dot to grow into local regions filled with the seed value until the growth encounters an adjacent region from an adjacent seed resulting in the expansion of the plurality of dots in the index image into a plurality of uniformly filled regions forming a Voronoi-type diagram in which each region is filled with the index value from a seed dot contained within that region;
f. create a local-maximum image by replacing each index value in the waterfall image with its corresponding value from the translation array resulting in a Voronoi-type diagram in which each region is uniformly filled with the maximum value of the source image for each region.
g. create a local threshold value in each of the plurality of regions by multiplying the local-maximum image by a threshold parameter with a range of [0.0 . . . 1.0] to create a local threshold value in each image region;
h. create a segmentation image wherein values are zero except where values in the source image region are larger than the values in the local-threshold image to isolate each local region into a single area which contains a maximum value of the region and any adjacent pixels which are above the local maximum value;
i. create a contour around the edge of each segmented image area;
j. create lists of outlines, wherein each outline has its own outline list of points and shape parameters, wherein each outline indicates the presence of a cell; and
k. count the number of cells.

In some embodiments of the apparatus the at least one processor is further configured for finding colonies of cells by counting the segmented outlines, enlarge each segmented outline and count outlines again; repeat the enlarging and counting until the total number of segmented outlines drops to a desired percentage of the starting count value; find contours that remain and determine the outline(s) of the region(s) which delineate where large colonies of cells are congregated, wherein the area enclosed by the outline(s) of the congregated colonie(s) are measured and compared to the total area of the subsection region to determine confluence.

As used herein, an “imager” refers to an imaging device for measuring light (e.g., transmitted or scattered light), color, morphology, or other detectable parameters such as a number of elements or a combination thereof. An imager may also be referred to as an imaging device. In certain embodiments, an imager includes one or more lenses, fibers, cameras (e.g., a charge-coupled device or CMOS camera), apertures, mirrors, light sources (e.g., a laser or lamp), or other optical elements. An imager may be a microscope. In some embodiments, the imager is a bright-field microscope.

As used herein, a “brightfield microscope” or “brightfield imager” is an imager that illuminates a sample and produces an image based on the light absorbed by or passing through the sample. Any appropriate bright-field microscope may be used in combination with an incubator provided herein. In some embodiments, the imager is outside of an incubator such as with the CellAssist, or an incubator cabinet includes a single imager as in the CellAssist50.

In some embodiments, an incubator cabinet includes two imagers.

Alternatively, the apparatus in some embodiments can include a display device such as a tablet mounted on one face of the system and the apparatus can operate as a self-contained unit.

In some embodiments, the image processing is either on board or external, has algorithms for artificial intelligence and intelligent image analysis. The image processing permits trend analysis and forecasting, documentation and reporting, live/dead cell counts, confluence percentage and growth rates, cell distribution and morphology changes, and the percentage of differentiation.

In some embodiments, one or more imaging systems may be interconnected by one or more networks in any suitable form, including as a local area network (LAN) or a wide area network (WAN) such as an enterprise network or the Internet. Such networks may be based on any suitable technology and may operate according to any suitable protocol and may include wireless networks, wired networks, or fiber optic networks.

When the imager is fully encased with the incubator, the interior of the imager can be set at 98.6 degrees F.° with a CO₂ content of 5%, so that the cells can remain in the imager without damage. The temperature and the CO₂ content of the air in the apparatus can be maintained by a gas feed. Alternatively, a heating unit can be installed in the apparatus to maintain the proper temperature. In some embodiments, the apparatus can also be connected to a computer or tablet for data input and output and for the control of the system. The connection can be by way of an ethernet connector or a USB connector.

In some embodiments, the imaging apparatus and method described herein can be used as a stand-alone imaging system or it can be integrated in a cell incubator using a transport described in the aforementioned application incorporated by reference. In some embodiments, the imaging system and method is integrated in a cell incubator and includes a transport.

In some embodiments the apparatus and method acquire data and images at the times a cell culturist typically examines cells. In some embodiments, the method and apparatus provide objective data, images, guidance and documentation that improves cell culture process monitoring and decision-making.

The apparatus and method in some embodiments enable sharing of best practices across labs, assured repeatability of process across operators and sites, traceability of process and quality control. In some embodiments the method and apparatus provide quantitative measures of cell doubling rates, documentation and recording of cell morphology, distribution and heterogeneity.

In some embodiments, the method and apparatus provide assurance that cell lines are treated consistently and that conditions and outcomes are tracked. Leveraging this database of observations, researchers are able to profile cell growth, test predictions and hypotheses concerning cell conditions, media and other factors affecting cell metabolism, and determine whether cells are behaving consistently and/or changing.

In some embodiments the method and apparatus enable routine and accurate cell counting and/or confluence measurements and imaging and enables biologists to quantify responses to stimulus or intervention, such as the administration of a therapeutic to a cell line.

The method and apparatus in some embodiments will capture the entire well area with higher coverage than conventional images and enables the highest level of statistical rigor for quantifying cell status and distribution.

In some embodiments, the method and apparatus provide additional image processing and algorithms that will deliver an integration of individual and group morphologies with process-flow information and biological outcomes. Full well imaging allows the analysis and modeling of features of groups of cells which are conducive to modeling organizational structures in biological development. These capabilities can be used for prediction of the organizational tendency of culture in advance of functional testing.

In some embodiments, algorithms are used to separate organizational patterns between samples using frequency of local slope field inversions. Using some algorithms, the method and system can statistically distinguish key observed differences between iP-MSCs generated from different TCP conditions. Biologically, this work could validate serum-free differentiation methods for iPSC MSC differentiation. Computationally, the method and apparatus can inform image-processing of MSCs in ways that less neatly “clustered” image sets are not as qualified to do. In some embodiments the method and apparatus described herein can be used to image IVF cells and tissue.

Even if all iP-MSC conditions have a sub-population of cells that meets ISCT 7-marker criteria, the “true MSC” sub-populations may occupy a different proportion under different conditions or fate differences could be implied by tissue “meso-structures”. By starting with a rich pallet of MSC outcomes, and grounding them in comparative biological truth, the method and apparatus can refine characterization perspectives around this complex cell type and improve MSC bioprocess.

As noted, cell cultures are composed of media, cells, and debris. The cells are volumes of protoplasm confined by a cell membrane into a compact region within the surrounding media. The optical properties of the protoplasm differ from the optical properties of the media. The media usually has a lower index of refraction than the cell contents, but the techniques described here can be easily adapted to the opposite relationship. The debris is frequently the residue of cells that have died and this confounds normal search techniques that seek to identify the living cells. In some embodiments the method and apparatus rely on the fact that the living cells have an intact surface membrane that continues to confine the internal content within a compact region.

The compact region, given sufficient space in the media, will pull itself into rounded shapes. These shapes, composed of fluids with a higher index of refraction, cause the rays of the illumination to be diverted toward the center of each such region much like a positive lens diverts light. This converges the illumination rays to create an image of increased brightness in a plane above the subject plane (above focus). In addition, if the camera lens is focused on a plane which is below the subject plane, the rays which would otherwise be available to image that plane “behind the compact region” have been diverted, creating a region of low brightness roughly in the shape of the compact region (below focus).

As cells are pushed together and touch each other as they grow, the boundaries tend to widen and appear as if they are not parts of the cells when they really are. To accommodate this, in some embodiments all non-cell areas are identified and their area is calculated. Related to the area between cells, an area threshold is defined based on a fixed area threshold, or a dynamic area threshold based on cell size, overall image confluence, neighborhood confluence and/or other measures. If the area between cells is below the threshold, that area is determined to be part of a cell or touching cells.

In some embodiments, each of the candidate cells are evaluated by their shape, color (or intensity), texture, contour features, etc. to determine whether they are really cells or if they are debris.

In some embodiments, all the areas defined as cells as a fraction of the total area where it is possible to grow cells are calculated as the confluence. In some embodiments, based upon cell type and density, we measure confluence in very confluent areas using one type of measuring method and in the less confluent areas we use a different type of measuring method. When we move away from the in-focus image in either direction, the size and shape of the bright/dark areas around the cells change in size. A dynamic method here would be to evaluate the size and shape of those dark/bright areas based on the expected size/shape for the cells of interest. When we move away from the in-focus image in either direction, the brightness/color/texture of the dark and light areas change depending on distance from focus. A dynamic method here would be to evaluate the brightness/color/texture of those dark/bright areas based on the expected brightness/color/texture for the cells of interest.

In some embodiments, a further step in the process includes alerting the user of the method and apparatus that confluence has reached a preselected value and that an action has to be taken such as passaging. In some embodiments, the steps above are used to perform cell counting.

As noted, the apparatus and method in some embodiments enable sharing of best practices across labs, assured repeatability of process across operators and sites, traceability of process and quality control. In some embodiments the method and system provide quantitative measures of cell doubling rates, documentation and recording of cell morphology, distribution and heterogeneity.

The apparatus and method in some embodiments capture the entire well area with higher coverage than conventional images and enables the highest level of statistical rigor for quantifying cell status and distribution.

In some embodiments, a further step in the process includes alerting the user of the method and apparatus that confluence has reached a preselected value and that an action has to be taken such as passaging. In some embodiments, the steps above are used to perform cell counting.

In some cases, operations such as controlling, operations of a cell culture incubator and/or components such as an imager provided therein or interfacing therewith, controlling the imager and components thereof, controlling a database and image processing, may be implemented using hardware, software or a combination thereof. When implemented in software, the software code can be executed on any suitable processor or collection of processors, whether provided in a single component or distributed among multiple components. Such processors may be implemented as integrated circuits with one or more processors in an integrated circuit component. The processor may be implemented using circuitry in any suitable format.

In some embodiments. a component (e.g., a controller) controls various processes performed inside the incubator, imager and image processing apparatus. For example, a controller may direct control equipment (e. g, a manipulator, an imager, a fluid handling, system. etc.). In some embodiments, the controller controls imaging of cell cultures, picking of cells. weeding of cells (e.g., removal of cell clumps) monitoring of cell culture conditions, adjustment of cell culture conditions, tracking of cell culture vessel movement within the incubator, comparing images, storing images maintaining a database and/or scheduling of any of the foregoing processes.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention.

More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used.

Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed.

The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, e.g., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (e.g. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, e.g., to mean including but not limited to.

Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for counting cells in image regions of a phase image, the source image, of a cell culture in a cell culture vessel, comprising the steps of:
a. find the local maximum of each source image region, wherein the size of each source image region is defined by an approximated diameter by creating an intermediate image using an elliptical structuring element mask having a deleted central pixel and performing a morphological operation of dilation on the source image;
b. for each source image region create a new mask image as a logical ‘LARGER THAN’ of the intermediate image with the source image resulting in zero everywhere except at a single pixel dot located at a pixel location wherein the source image has a local maximum value of a local region and wherein the size of the local region is determined by the size of the elliptical structuring element mask resulting in the mask image having a multitude of single pixel dots.
c. create an index image wherein each single non-zero pixel of the mask image is replaced by a unique index value beginning with one and increasing by one for each additional non-zero pixel value;
d. using the index image to create a translation array of source image values, one for each index in the index image, in which the value of the translation array is set to the value contained in the source image at a pixel location identical to the pixel location of each index value of the index image;
e. create a waterfall image using the index image as a seed image causing each seed dot to grow into local regions filled with the seed value until the growth encounters an adjacent region from an adjacent seed resulting in the expansion of the plurality of dots in the index image into a plurality of uniformly filled regions forming a Voronoi-type diagram in which each region is filled with the index value from a seed dot contained within that region;
f. create a local-maximum image by replacing each index value in the waterfall image with its corresponding value from the translation array resulting in a Voronoi-type diagram in which each region is uniformly filled with the maximum value of the source image for each region.
g. create a local threshold value in each of the plurality of regions by multiplying the local-maximum image by a threshold parameter with a range of [0.0 . . . 1.0] to create a local threshold value in each image region;
h. create a segmentation image wherein values are zero except where values in the source image region are larger than the values in the local-threshold image to isolate each local region into a single area which contains a maximum value of the region and any adjacent pixels which are above the local maximum value;
j. create a contour around the edge of each segmented image area;
k. create lists of outlines, wherein each outline has its own outline list of points and shape parameters, wherein each outline indicates the presence of a cell; and
l. count the number of cells.

2. The method of claim 1, further comprising finding colonies of cells by counting the segmented outlines, enlarge each segmented image area, recreate the outlines, and count outlines again; repeat the enlarge and count steps until the total number of segmented outlines drops to a desired percentage of the starting count value; find contours that remain and determine the outline(s) of the region(s) which delineate where large colonies of cells are congregated, wherein the area enclosed by the outline(s) of the congregated colonie(s) are measured and compared to the total area of the subsection region to determine confluence.

3. An apparatus for counting cells in image regions of a phase image of a cell culture in a cell culture vessel, comprising: an imager for imaging a cell culture in a cell culture vessel; and at least on processor for providing a phase image of the cell culture and configured to:
a. find the local maximum of each source image region, wherein the size of each source image region is defined by an approximated diameter by creating an intermediate image using an elliptical structuring element mask having a deleted central pixel and performing a morphological operation of dilation on the source image;
b. for each source image region create a new mask image as a logical ‘LARGER THAN’ of the intermediate image with the source image resulting in zero everywhere except at a single pixel dot located at a pixel location wherein the source image has a local maximum value of a local region and wherein the size of the local region is determined by the size of the elliptical structuring element mask resulting in the mask image having a multitude of single pixel dots.
c. create an index image wherein each single non-zero pixel of the mask image is replaced by a unique index value beginning with one and increasing by one for each additional non-zero pixel value;
d. using the index image to create a translation array of source image values, one for each index in the index image, in which the value of the translation array is set to the value contained in the source image at a pixel location identical to the pixel location of each index value of the index image;
e. create a waterfall image using the index image as a seed image causing each seed dot to grow into local regions filled with the seed value until the growth encounters an adjacent region from an adjacent seed resulting in the expansion of the plurality of dots in the index image into a plurality of uniformly filled regions forming a Voronoi-type diagram in which each region is filled with the index value from a seed dot contained within that region;
f. create a local-maximum image by replacing each index value in the waterfall image with its corresponding value from the translation array resulting in a Voronoi-type diagram in which each region is uniformly filled with the maximum value of the source image for each region.
g. create a local threshold value in each of the plurality of regions by multiplying the local-maximum image by a threshold parameter with a range of [0.0 . . . 1.0] to create a local threshold value in each image region;
h. create a segmentation image wherein values are zero except where values in the source image region are larger than the values in the local-threshold image to isolate each local region into a single area which contains a maximum value of the region and any adjacent pixels which are above the local maximum value;
i. create a contour around the edge of each segmented image area;
j. create lists of outlines, wherein each outline has its own outline list of points and shape parameters, wherein each outline indicates the presence of a cell; and
k. count the number of cells.

4. The apparatus of claim 3, wherein the at least one processor is further configured for finding colonies of cells by counting the segmented outlines, enlarge each segmented image area, recreate the outlines, and count outlines again; repeat the enlarging and counting until the total number of segmented outlines drops to a desired percentage of the starting count value; find contours that remain and determine the outline(s) of the region(s) which delineate where large colonies of cells are congregated, wherein the area enclosed by the outline(s) of the congregated colonie(s) are measured and compared to the total area of the subsection region to determine confluence.
